SEQUENCE LISTING (13 sequences)

SEQ ID NO: 1 - DNA, 20 bp, Artificial Sequence
  Primer
agagtttgatcmtggctcag

SEQ ID NO: 2 - DNA, 22 bp, Artificial Sequence
  Primer
tacggytaccttgttacgactt

SEQ ID NO: 3 - DNA, 1362 bp, Enterococcus faecium
aaaaagaggagtggcgaacgggtgagtaacacgtgggtaacctcgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtga

SEQ ID NO: 4 - DNA, 1391 bp, Enterococcus faecium
cgcttctttttccaccggagcttgctccaccggaaaaagaagagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcccccccccaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcgg

SEQ ID NO: 5 - DNA, 1352 bp, Enterococcus faecium
aagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagt

SEQ ID NO: 6 - DNA, 1388 bp, Enterococcus faecium
gcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtc

SEQ ID NO: 7 - DNA, 1389 bp, Enterococcus faecium
ggagcttgctccaccggaaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttt

SEQ ID NO: 8 - DNA, 1358 bp, Enterococcus faecium
gaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcg

SEQ ID NO: 9 - DNA, 1372 bp, Enterococcus faecium
ttctttttccaccggagcttgctccaccggaaaaagaagagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttg

SEQ ID NO: 10 - DNA, 1381 bp, Enterococcus faecium
ggagcttgcttccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgagg

SEQ ID NO: 11 - DNA, 20 bp, Artificial Sequence
  primer
agagtttgatcmtggctcag

SEQ ID NO: 12 - DNA, 22 bp, Artificial Sequence
  primer
tacggytaccttgttacgactt

SEQ ID NO: 13 - DNA, 1339 bp, Enterococcus faecalis
agtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcc

(19) United States
(12) Patent Application Publication
(10) Pub. No.: US 2025/0312393 A1
(43) Pub. Date: Oct. 9, 2025

(54) ANTITUMOR BACTERIAL STRAIN, AND COMPOSITION AND METHOD USING SAME

(71) Applicant: LIVEOME INC., Suwon-si, Gyeonggi-do (KR)

(72) Inventors: Yeung Hyen KIM, Hwaseong-si, Gyeonggi-do (KR); Do Hyeong KWON, Suwon-si, Gyeonggi-do (KR); Kwang Seo PARK, Suwon-si, Gyeonggi-do (KR); Hye Jin DONG, Incheon (KR); Hye Jeong JANG, Seoul (KR); Ji Yoon SONG, Seongnam-si, Gyeonggi-do (KR)

(21) Appl. No.: 18/023,492

(22) PCT Filed: Aug. 9, 2021
(86) PCT No.: PCT/KR2021/010525

§ 371 Date: Feb. 27, 2023

(30) Foreign Application Priority Data
Aug. 28, 2020 (KR) 10-2020-0109411
Sep. 23, 2020 (KR) 10-2020-0123330

(51) Int. Cl. A61K 35/744; A61K 39/00; A61P 35/00; C07K 16/28; C12N 9/42

(52) U.S. Cl. CPC A61K 35/744; A61P 35/00; C07K 16/2818; C07K 16/2827; C12N 9/2445; A61K 2039/505; C12Y 302/01023

(57) ABSTRACT

Provided are Enterococcus faecium microorganisms or Enterococcus faecalis microorganisms having anti-cancer activity, or a composition containing the same, and a method of preventing or treating cancer by using the composition.

### TECHNICAL FIELD

The present disclosure relates to anti-tumor bacterial strains, and compositions and methods using the same, more specifically, bacterial strains of a species of Enterococcus faecium exhibiting anti-tumor activity, and compositions and methods for preventing or treating tumors by using the same.

In addition, the present disclosure relates to anti-tumor bacterial strains, and compositions and methods using the same, and more specifically, to Enterococcus faecalis microorganisms having anti-cancer activity, a composition containing the same, and a method of preventing or treating cancer by using the same.

### BACKGROUND ART

It is known that gut bacteria may be used to prevent or treat various diseases or disorders.

International Publication No. WO2016/196605 discloses a method of treating or preventing cancer in a subject, by modulating a level of one or more commensal microbes in the subject to: enhance an immune response by the subject; and/or inhibit growth or spread of cancer; and/or inhibit immune evasion of cancer; and/or enhance efficacy of a therapeutic agent. This reference cites genera Adlercreutzia, Oscillopira, Mollicutes, Butyrivibrio, Bacteroides, Clostridium, Fusobacterium, Eubacterium, Ruminococcus, Peptococcus, Peptostreptococcus, Bifidobacterium, Rikenella, Alistipes, Marinilabilia, Anaerostipes, Escherichia, Lactobacillus as examples of commensal microbes, and discloses a method of treating cancer by using bacteria of a genus Bifidobacterium in combination with an immune checkpoint inhibitor.

International Publication No. WO2017/085520 discloses a composition for use in a method for treating or preventing cancer, including a bacterial strain of a species Enterococcus gallinarum.

International Publication No. WO2017/085518 discloses a composition for use in a method of treating or preventing a disease or condition mediated by the IL-17 or Th17 pathway, including a bacterial strain of a species Enterococcus faecium.

To date, correlation between anti-tumor activity and an ability to produce a specific metabolite, compared to microbial growth in Enterococcus faecium species has not been known.

In addition, Enterococcus faecalis is found in gastrointestinal tracts of humans and other mammals. Enterococcus faecalis is a gram-positive, cocci bacterium. Enterococcus faecalis can grow at a temperature of 10° C. to 45° C.

Korean Patent Publication No. 10-2020-0054589 discloses Enterococcus faecalis KACC 92220P having an effect of lowering a lactose content. In addition, Korean Patent No. 10-2053730 discloses an Enterococcus faecalis AMI-1001 strain having anti-oxidant, anti-inflammatory, or anti-bacterial activity.

However, even according to the above-described related art, there is a demand for Enterococcus faecalis having anti-cancer activity.

### DESCRIPTION OF EMBODIMENTS

### Technical Problem

A first object of the present disclosure is to provide a bacterial strain of a species Enterococcus faecium that has anti-tumor activity, which is characterized by having a lactate production ability compared to microbial growth.

A second object of the present disclosure is to provide a composition for preventing or treating tumors, including the bacterial strain of the Enterococcus faecium species as an active ingredient.

A third object of the present disclosure is to provide a method of preventing or treating tumors in a subject, including administering the bacterial strain of the Enterococcus faecium species or the composition to the subject.

A fourth object of the present disclosure is to provide Enterococcus faecalis LMT19-32 (accession number KCTC 14306BP) microorganisms, which have anti-tumor activity, or a culture or an extract thereof.

A fifth object of the present disclosure is to provide a pharmaceutical composition for preventing or treating cancer containing the Enterococcus faecalis LMT19-32 microorganisms or a culture or an extract thereof as an active ingredient.

A sixth object of the present disclosure is to provide a food composition for preventing or ameliorating cancer containing the Enterococcus faecalis LMT19-32 microorganisms or a culture or an extract thereof as an active ingredient.

A seventh object of the present disclosure is to provide a method of preventing or treating cancer in a subject including administering to a subject an effective amount of the Enterococcus faecalis LMT19-32 microorganisms or a culture or extract thereof for treating cancer.

### Solution to Problem

A first aspect of the disclosure provides a bacterial strain that
i) belongs to a species Enterococcus faecium,
ii) has a lactate production ability compared to microbial growth (lactate/OD₆₀₀) of 3 g/L or more, when cultured for 48 hours, and
iii) exhibits anti-tumor activity.

A second aspect of the present disclosure provides a composition for preventing or treating tumor, including the bacterial strain of the Enterococcus faecium species as an active ingredient.

A third aspect of the present disclosure is to provide a method of preventing or treating cancer in a subject, including administering to a subject the bacterial strain of the species of Enterococcus faecalis, or the composition.

A fourth aspect of the present disclosure is to provide Enterococcus faecalis LMT19-32 (accession number KCTC 14306BP) microorganisms, which have anti-tumor activity, or a culture or an extract thereof.

A fifth aspect of the present disclosure is to provide a pharmaceutical composition for preventing or treating cancer, containing the Enterococcus faecalis LMT19-32 microorganisms or a culture or an extract thereof as an active ingredient.

A sixth aspect of the present disclosure is to provide a food composition for preventing or ameliorating cancer, containing the Enterococcus faecalis LMT19-32 microorganisms or a culture or an extract thereof as an active ingredient.

A seventh aspect of the present disclosure is to provide a method of preventing or treating cancer in a subject including administering to a subject an effective amount of the Enterococcus faecalis LMT19-32 microorganisms or a culture or extract thereof for treating cancer.

### Advantageous Effects of Disclosure

The bacterial strain according to the present disclosure increases numbers of tumor-infiltrating T cells, CD8 T cells, and IFN_{γ+} CD8 T cells, and thus exhibits excellent tumor suppression activity, and therefore may be effectively used to prevent or treat tumors.

In another aspect, according to the Enterococcus faecalis LMT19-32 (accession number KCTC 14306BP) microorganisms having anti-tumor activity or a culture or extract thereof according to the present disclosure, cancer may be prevented or treated.

In another aspect, according to the pharmaceutical composition for preventing or treating cancer according to the present disclosure, cancer may be prevented or treated.

In another aspect, according to the food composition for preventing or ameliorating cancer according to the present disclosure, caner may be prevented or ameliorated.

In another aspect, according to the method of preventing or treating cancer in a subject according to the present disclosure, cancer may be efficiently prevented or treated.

### BRIEF DESCRIPTION OF DRAWINGS

FIG. 1 shows optical microscope images of a strain Enterococcus faecium LMT17-62 and a type strain KCTC13225 according to an embodiment of the present disclosure.

FIG. 2A is a graph showing tumor growth inhibition rates (%) of Enterococcus faecium strains selected from mouse tumor induction models.

FIG. 2B are graphs showing a change in a tumor size over time in a group administered with the strain Enterococcus faecium LMT17-62.

FIG. 2C are graphs showing changes in numbers of total tumor-infiltrating T cells, CD8 T cells, and interferon gamma-positive CD8 T cells in the group administered with the strain Enterococcus faecium LMT17-62.

FIG. 3A is a growth (OD₆₀₀) curve of a selected Enterococcus faecium strain.

FIG. 3B is a graph showing an amount of lactate produced according to incubation time of the selected Enterococcus faecium strain.

FIG. 3C are graphs showing amounts of lactate production (g/L) as compared to growth (OD₆₀₀) of selected Enterococcus faecium strains after culturing for 24 hours and 48 hours.

FIG. 4 is a graph showing numbers of live bacteria of the selected Enterococcus faecium strains when the strains are cultured at pH 2.5.

FIG. 5 is a graph showing numbers of live bacteria of the selected Enterococcus faecium strains when the strains are cultured in a bile salt-containing medium.

FIG. 6 is a graph showing numbers of live bacteria of the selected anti-tumor-positive Enterococcus faecium strains attached to intestinal epithelial cells.

FIG. 7 shows representative optical microscope images of a selected strain Enterococcus faecalis LMT19-32 and a type strain KCTC3206.

FIGS. 8A and 8B are diagrams showing results of measuring tumor sizes after administering the Enterococcus faecalis LMT19-32 strain to mice with tumor.

FIGS. 9A, 9B, and 9C show results of analyzing tumor-infiltrating T cells, CD8 T cells, and IFN_{γ+} CD8 T cells after administration of the Enterococcus faecalis LMT19-32 strain to mice with tumor.

### BEST MODE

### 1. Enterococcus faecium

The present disclosure is based on a finding that bacterial strains of the species Enterococcus faecium, which are characterized by their ability to produce lactate compared to microbial growth, exhibit excellent anti-tumor activity.

The terms “tumor” and “cancer” are used interchangeably and encompass solid and liquid, for example, diffuse or circulating tumors. The terms include precancer as well as malignant cancers and tumors. The terms also include primary malignant cells or tumors and secondary malignant cells or tumors (for example, metastatic tumors).

The terms “anti-tumor” and “anti-cancer” may refer to biological effects that may be expressed by a variety of means, including but not limited to, for example, reduction in tumor size, reduction in a number of tumor cells, reduction in tumor cell proliferation, or reduction in tumor cell survival.

An aspect of the disclosure provides a bacterial strain that
i) belongs to a species Enterococcus faecium,
ii) has a lactate production ability compared to microbial growth (lactate/OD₆₀₀) of 3 g/L or more, when cultured for 48 hours, and
iii) exhibits anti-tumor activity.

In an embodiment, the bacterial strain may have a lactate production ability (lactate/OD₆₀₀) compared to microbial growth of greater than 2.5 g/L, when the bacterial strain is cultured for 24 hours.

In an embodiment, the bacterial strain may exhibit a tumor growth inhibition rate of 10% or more, 15% or more, 20% or more, 25% or more, 30% or more, or 35% or more. In a certain example, the bacterial strain may exhibit a tumor growth inhibition rate of about 10% to about 40%, about 15% to about 40%, about 20% to about 40%, about 25% to about 40%, about 30% to about 40%, or about 35% to about 40%.

In an embodiment, the bacterial strain may have iv) β-galactosidase activity.

In an embodiment, the bacterial strain may have v) a D-sorbitol decomposition ability.

In an embodiment, the bacterial strain may have vi) a D-tagatose decomposition ability.

In an embodiment, the bacterial strain may have vii) a methyl-αD-mannopyranoside decomposition ability.

In an embodiment, the bacterial strain may satisfy the three characteristics of i) to iii), the four characteristics of i) to iv), five characteristics of i) to v), i) to iv), i) to vi), or i) to vii), six characteristics of i) to vi), or i) to vii), or seven characteristics of i) to vii).

In a specific example, the bacterial strain may be one or more of those shown in Table 1 below, but is not limited thereto.

TABLE 1

| | | Characteristics | | | | |
|---|---|---|---|---|---|---|
| | Accession | Lactate/ | β- | D- | D- | Methyl-αD- |
| Name | number | OD₆₀₀(g/L) | galactosidase | sorbitol | tagatose | mannopyranoside |
| LMT17- | KCTC | >3 | + | + | + | + |
| 62 | 14284BP | | | | | |
| LMT17- | KCTC | >3 | + | + | + | + |
| 74 | 14285BP | | | | | |
| LMT15- | KCTC | >3 | + | + | + | − |
| 24 | 14289BP | | | | | |
| LMT17- | KCTC | >3 | + | − | − | − |
| 25 | 14288BP | | | | | |
| LMT15- | KCTC | >3 | − | − | − | − |
| 4 | 14290BP | | | | | |

A second aspect of the present disclosure relates to a composition for preventing or treating tumors, comprising the bacterial strain as an active ingredient. The terms “treat,” “treating,” or “treatment” refer to, for example, healing injured or damaged tissue, achieving desired therapeutic results by altering, changing, strengthening, ameliorating, improving, and/or beautifying a pre-existing or recognized disease, disorder, or condition; or alleviating, reducing (including partial reduction, substantial reduction, near complete reduction, and complete reduction), resolving or preventing (whether temporarily or permanently) of a disease, a disorder, or a condition.

The term “prevention” means delaying an onset of a disease, disorder, or condition. Prevention may be considered complete when an onset of a disease, disorder, or condition is delayed for a pre-determined period of time.

In an embodiment, the bacterial strain included in the composition may exist as live bacteria or dead bacteria, and may exist in a dried or lyophilized form.

In an embodiment, the bacterial strain may be used in any form, such as a culture or an isolated strain, as long as the strain retains its anti-tumor activity, and all forms fall within the scope of the present disclosure. The term “culture” refers to a composition including a cultured strain, its metabolites, and extra nutrients, etc., obtained by culturing the strain for a certain period of time in a medium that can supply nutrients so that the bacterial strain of the present disclosure may grow and survive in vitro.

In an embodiment, the composition may be for oral, or parenteral administration. Parenteral administration includes, but is not limited to, intravenous injection, subcutaneous injection, intramuscular injection, intraperitoneal injection, intradermal administration, topical administration, intranasal administration, intrapulmonary administration, intrarectal administration, intratumoral administration, and the like. In a specific example, the composition may be for oral administration.

The composition may be a pharmaceutical composition, in which case the pharmaceutical composition may be formulated by further including a pharmaceutically acceptable carrier or additive, in addition to the active ingredient. The carrier or additive may include an excipient, a disintegrant, a sweetener, a binder, a coating agent, an expanding agent, a lubricant, a glydent, a flavoring agent, a coloring agent, a diluent, a dispersing agent, a surfactant, an anti-oxidant, a buffer, a bacteriostatic agent, and the like. The composition may be formulated into a pill, a powder, a capsule, a granule, or a tablet, or an injectable formulation such as an aqueous solution, suspension, or emulsion.

The composition may be a food or a food additive composition, in which case the composition may include a sitologically acceptable diluent or carrier. The diluent may be water, medium or a buffer such as PBS. The carrier may be a commonly used excipient, disintegrant, binder, glydent, thickener, or filler. The food may be a health functional food. The food may be a beverage, confectionery, diet bar, chocolate, pizza, ramen, other noodles, chewing gum, ice cream, and the like.

When the composition is for oral administration, it may be coated with a coating agent to increase acid resistance, thermal resistance, bile resistance, survival rate, intestinal anchorage, etc. of the bacterial strain. A coating agent that may be used include, but is not particularly limited to, enteric coating agents; gelatin, polysaccharides, gums and the like; water soluble polymers, hyaluronic acid, porous particles, and proteins; casein, and coating agent, edible fat and oil, extracellular polymeric substance of Lactobacillus plantarum, and alginic acid; silk fibroin, and the like, and the coating may be a single coating or multiple coatings, for example, double, triple, or quadruple coatings.

In an embodiment, the tumor may be a solid tumor. Non-limiting examples of the solid tumor include, but are not limited to, breast cancer, lung cancer, head or neck cancer, colorectal cancer, esophageal cancer, laryngeal cancer, stomach cancer, liver cancer, pancreatic cancer, bone cancer, skin cancer, skin or intraocular melanoma, uterine cancer, ovarian cancer, rectal cancer, proximal anal cancer, colon cancer, breast cancer, fallopian tube carcinoma, endometrial carcinoma, cervical carcinoma, vaginal carcinoma, vulvar carcinoma, Hodgkin's disease, small intestine cancer, endocrine cancer, thyroid cancer, parathyroid cancer, adrenal cancer, soft tissue sarcoma, urethral cancer, penile cancer, prostate cancer, lymphocytic lymphoma, bladder cancer, renal or ureteric cancer, renal cell carcinoma, renal pelvic carcinoma, CNS tumor, primary CNS lymphoma, spinal cord tumor, brainstem glioma, and pituitary adenoma.

In an embodiment, a dosage may vary depending on the patient's body weight, age, sex, health condition, diet, excretion rate, and constitutional specificity, administration time administration method, administration period or interval, nature of the formulation, severity of the disease, etc. and may be appropriately selected by a person skilled in the art. For example, the bacterial strain, which is an active ingredient, may be administered in an amount of 1×10⁶ CFU or more, 1×10⁷ CFU or more, 1×10⁸ CFU or more, 1×10⁹ CFU or more. For example, the bacterial strain may be administered in an amount of 1×10¹⁵ CFU or less, 1×10¹⁴ CFU or less, 1×10¹³ CFU or less, 1×10¹² CFU or less. For example, the bacterial strain may be administered in an amount of about 1×10⁶ to about 1×10¹⁵ CFU, about 1×10⁷ to about 1×10¹⁴ CFU, about 1×10⁸ to about 1×10¹³ CFU, about 1×10⁹ to about 1×10¹² CFU, about 1×10¹⁰ to about 1×10¹² CFU, or about 1×10¹¹ to about 1×10¹² CFU. For example, the bacterial strain, which is an active ingredient, may be administered once a day or several times a day in aliquots.

In an embodiment, the composition may be for using in combination with one or more other therapeutic agents, for example, anti-cancer agents, anti-viral agents, cytokines, or immuneagents.

The term “use in combination” refers to any form of administration of two or more different therapeutic agents such that a second therapeutic agent is administered while a previously administered therapeutic agent is still effective in the body. For example, two therapeutic agents are simultaneously effective in a subject, and there may be a synergistic effect of the two therapeutic agents. The different therapeutic agents may be administered concurrently or sequentially in a single formulation or in separate formulations.

In a specific example, the anti-cancer agent may be a chemotherapeutic agent. Non-limiting examples of the chemotherapeutic agents include alkylating agents, nitrosoureases, anti-metabolites, anti-cancer anti-biotics, plant-derived alkaloids, topoisomerase inhibitors, hormonal drugs, hormone antagonists, leukopenia (neutropenia) treatment drugs, thrombocytopenia drugs, anti-emetics, aromatase inhibitors, P-glycoprotein inhibitors, platinum complex derivatives, and other immunotherapy drugs and other anti-cancer drugs. Cytotoxic agents that may be co-administered include, for example, anti-microtubule agents, topoisomerase inhibitors, anti-metabolites, mitotic inhibitors, alkylating agents, anthracyclines, vinca alkaloids, intercalating agents, agents that may interfere with signal transduction pathways. agents that promote apoptosis, proteasome inhibitors, and radiation (local or systemic irradiation). Non-limiting examples of additional therapeutic agents include, but are not limited to, peptides, polypeptides, proteins, fusion proteins, nucleic acid molecules, small molecules, mimetic agents, synthetic drugs, inorganic and organic molecules.

In a specific example, the immuno-anti-cancer agent may be a chemotherapeutic agent. The term “immuno-anti-cancer agent” refers to a compound, composition or treatment that indirectly or directly enhances, stimulates or increases the body's immune response to cancer cells and/or reduces side effects of other anti-cancer therapies. Non-limiting examples of immuno-anti-cancer agents include cytokines, cancer vaccines, monoclonal antibodies, non-cytokine adjuvants, immune cells (T cells, NK cells, dendritic cells, B cells, etc.), immune checkpoint inhibitors, and the like. In a specific example, the immuno-anti-cancer agent is an immune checkpoint inhibitor. Immune checkpoint inhibitors include peptides, antibodies, nucleic acid molecules, and small molecules. For example, an immune checkpoint inhibitor may be administered to enhance proliferation, migration, persistency and/or cytotoxic activity of CD8+ T cells in a subject, and in particular, tumor infiltration of CD8+ T cells in a subject. Typically, immune checkpoint inhibitors target activated T lymphocytes, such as cytotoxic T lymphocyte-associated protein 4 (CTLA4) and programmed cell death 1 (PD-1), or various members of the killer cell immunoglobulin-like receptor (KIR) family, which are antagonists that block immunosuppressive receptors expressed by NK cells, or antagonists that block key ligands of the receptor, for example, PD-1 ligand CD274 (best known as PD-L1 or B7-H1). For example, an immune checkpoint inhibitor is an antibody or an antigen-binding fragment thereof. Specifically, an immune checkpoint inhibitor may be at least one selected from the group consisting of anti-PD-1 antibodies, anti-PD-L1 antibodies, anti-PD-L2 antibodies, anti-CTLA-4 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies, and antigen-binding fragments thereof. More specifically, an immune checkpoint inhibitor may be at least one selected from ipilimumab (Yervoy®, BMS/Ono), tremelimumab (AstraZeneca), atezolizumab (Tecentriq®, Roche), nivolumab (Opdivo®, BMS/Ono), pembrolizumab (Keytruda®, MSD), avelumab (Bavencio®, Pfizer/Merck, Germany), durvalumab (Imfinzi®, AstraZeneca/Medimmune), and antigen-binding fragments thereof, but is not limited thereto.

In an embodiment, the immuno-anti-cancer agent may be formulated and used in various forms suitable for each purpose of use according to a method commonly used in the art, for example, as an oral formulation such as a liquid, suspension, powder, granule, tablet, capsule, pill, extract, emulsion, syrup, and aerosol, and a parenteral formulation such as an injection of a sterile injection solution. Immuno-anti-cancer agents may be administered orally or parenterally through various routes, including intravenous, intraperitoneal, subcutaneous, intradermal, intramuscular, spinal, intrathecal, rectal local administration, or injection. A dosage may vary depending on the patient's body weight, age, sex, health condition, diet, excretion rate, and constitutional specificity, administration time, administration method, administration period or interval, nature of the formulation, severity of the disease, etc. and may be appropriately selected by a person skilled in the art. For example, the dosage may be in a range of about 0.1 mg/kg to about 10,000 mg/kg, but is not limited thereto, and may be administered once a day or several times a day in aliquots.

A third aspect of the present disclosure provides a method of preventing or treating cancer in a subject, including administering to a subject the bacterial strain, or the composition.

The term “administration” or “administering” means a process of providing an active ingredient or a composition including the same to a subject. The active ingredient or the composition including the same may be administered through various appropriate routes.

Subjects to be administered may include humans or animals, for example, humans, pigs, dogs, cats, cows, horses, mice, and the like without limitation.

### II. Enterococcus faecalis LMT19-32

The first aspect of the present disclosure provides Enterococcus faecalis LMT19-32 (accession number KCTC 14306BP) microorganisms, which have anti-tumor activity, or a culture or an extract thereof.

The microorganisms, or the culture or the extract thereof may inhibit tumor growth. The microorganisms, or the culture or the extract thereof may increase a level of immune cells in a tumor. The microorganisms, or the culture or the extract thereof may increase a level of CD8 T cells in a tumor, for example, CD8 T cells expressing IFNγ. The microorganisms, or the culture or the extract thereof may increase a level of infiltration of immune cells into a tumor. The immune cells may be CD8 T cells, CD4 T cells, NK cells, B cells, dendritic cells, macrophages, and neutrophils. The microorganisms, or the culture or the extract thereof may activate immune cells. The activation may be such that the microorganisms, or the culture or the extract thereof promotes production, secretion, or production and secretion of cytokines or enzymes having anti-tumor activity. The cytokine or enzyme may be one or more of interferon gamma and granzyme B. The microorganisms are excellent in bile resistance and intestinal anchorage. The microorganisms are isolated from human feces.

When the microorganisms are cultured in De Man, Rogosa and Sharpe agar (MRS) medium containing 0.3% of bile acid for 2 hours at 37° C., the bile acid resistance may be 65% or more, 70% or more, 75% or more, 80% or more, 85% or more, 90% or more, 93% or more, 95% or more, about 75% to about 90%, about 75% to about 95%, about 80% to about 90%, about 80% to about 95%, about 85% to about 90%, or about 90% to about 95% of survival rate.

The microorganisms, or the culture or the extract thereof may promote infiltration of CD8 T cells into tumor. The promotion may increase a percentage of CD8 T cells to the number of T cells in the tumor by 5% or more, 10% or more, 15% or more, 20% or more, 25% or more, 30% or more, 35% or more, 45% or more, 50% or more, 55% or more, 65% or more, 70% or more, 75% or more, 80% or more, 85% or more, 90% or more, 100% or more, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, or about 90% to about 100%, compared to a case in which the microorganisms, or the culture or the extract thereof is not present.

The microorganisms, or the culture or the extract thereof may promote production, secretion, or production and secretion of one or more of interferon gamma and granzyme B in tumor-infiltrating CD8 T cells. The promotion may increase a percentage of cells producing interferon gamma among CD8 T cells by 5% or more, 10% or more, 15% or more, 20% or more, 25% or more, 30% or more, 35% or more, 45% or more, 50% or more, 55% or more, 65% or more, 70% or more, 75% or more, 80% or more, 85% or more, 90% or more, 100% or more, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, or about 90% to about 100%, compared to the case in which the microorganisms, or the culture or the extract thereof is not present. In addition, the promotion may increase a percentage of cells producing granzyme B among CD8 T cells by 5% or more, 10% or more, 15% or more, 20% or more, 25% or more, 30% or more, 35% or more, 45% or more, 50% or more, 55% or more, 65% or more, 70% or more, 75% or more, 80% or more, 85% or more, 90% or more, 100% or more, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, or about 90% to about 100%, compared to the case in which the microorganisms, or the culture or the extract thereof is not present.

The microorganisms, or the culture or the extract thereof may suppress growth of a tumor, by CD8 T cells. The suppression may decrease a tumor size by 5% or more, 10% or more, 15% or more, 20% or more, 25% or more, 30% or more, 35% or more, 45% or more, 50% or more, 55% or more, 65% or more, 70% or more, 75% or more, 80% or more, 85% or more, 90% or more, 100% or more, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, or about 90% to about 100%, compared to a case in which the microorganisms, or the culture or the extract thereof is not present.

A second aspect provides a pharmaceutical composition for preventing or treating cancer including the microorganisms, or the culture or the extract thereof as an active ingredient.

In the pharmaceutical composition, the cancer may be a solid cancer. The cancer may be a solid cancer existing in a tissue other than a tissue directly contacting the microorganisms when the microorganisms are orally administered. The tissues in direct contact with the microorganisms include the oral cavity, esophagus, stomach, duodenum, small intestine, large intestine, and colon. In addition, tissues other than the tissues in direct contact are breast, lung, head, neck, liver, pancreas, bone, fallopian tube, uterus, vagina, vulva, thyroid, parathyroid, adrenal, soft tissue, urethra, penis, prostate, bladder, kidney, ureter, or central nervous system (CNS). The cancer may be metastatic cancer. The cancer may be, for example, cancers listed in “I. Enterococcus faecium”, but are not limited thereto.

In the pharmaceutical composition, the composition may be for inhibiting growth of cancer.

In the pharmaceutical composition, the composition may be for co-administering with an immune checkpoint inhibitor. The immune checkpoint inhibitor may be for administering before, concurrently with, or after administration of the microorganisms, or the culture or the extract thereof.

The immune checkpoint inhibitor may be, for example, those listed in “I. Enterococcus faecium”, but is not limited thereto.

In the pharmaceutical composition, the composition may be for co-administering with a chemotherapeutic agent. The chemotherapeutic agent may be for administering before, concurrently with, or after the administration of the microorganisms, or the culture or the extract thereof.

The chemotherapeutic agent may be, for example, those listed in “I. Enterococcus faecium”, but is not limited thereto.

In the pharmaceutical composition, the composition may include a pharmaceutically acceptable carrier. The carrier may be a stabilizer, excipient, diluent, or adjuvant. The carrier may be, for example, any and all aqueous and non-aqueous solutions, sterile solutions, solvents, buffers such as a phosphate buffered saline (PBS) solution, water, suspensions, emulsions such as oil/water emulsions, various types of wetting agents, liposomes, dispersion media and coating agents suitable for pharmaceutical administration, particularly suitable for oral administration. The use of such media and agents in pharmaceutical compositions is well known in the art, and compositions including such carriers may be formulated by a well known and common method in the art.

The composition may contain the microorganisms, or the culture or the extract thereof in a “therapeutically effective amount”. In the composition, “therapeutically effective amount” means an amount sufficient to exhibit a therapeutic effect when administered to a subject in need of treatment when administered once, or twice or more times. The term “treatment” means treating a cancer disease or medical symptoms of cancer in a mammal, including a human, and includes: alleviating a cancer disease or medical symptoms of cancer; inhibiting a cancer disease or medical symptoms of cancer, that is, slowing or stopping progression of a disease or medical symptoms in a subject; or ameliorating a cancer disease or medical symptoms of cancer in a subject. The “effective amount” may be appropriately selected by a person skilled in the art. The “effective amount” may be about 0.01 wt % to about 50 wt %, or about 0.1 wt % to about 20 wt %, with respect to the weight of the composition. In addition, an amount of the composition administered may be 1×10⁶ CFU/g or more, 1×10⁷ CFU/g or more, 1×10⁸ CFU/g or more, or 1×10⁹ CFU/g or more, with respect to the weight of the composition. For example, the bacterial strain may be administered in an amount of 1×10¹⁵ CFU or less, 1×10¹⁴ CFU or less, 1×10¹³ CFU or less, or 1×10¹² CFU or less. For example, the bacterial strain included in the composition may be about 1×106 CFU/g to about 1×10¹⁵ CFU/g, about 1×10⁷ CFU/g to about 1×10¹⁴ CFU/g, about 1×10⁸ CFU/g to about 1×10¹³ CFU/g, about 1×10⁹ CFU/g to about 1×10¹² CFU/g, or about 1×10¹⁰ CFU/g to about 1×10¹² CFU/g.

The composition may be administered orally. Accordingly, the composition may be formulated in various forms such as tablets, capsules, liquid formulations such as aqueous solutions, dry syrups or suspensions. For tablets for oral administration, excipients such as lactose and corn starch, and lubricants such as magnesium stearate may be usually added. For capsules for oral administration, lactose and/or dried corn starch may be used as diluents. When oral aqueous suspensions are required, an active ingredient may be combined with emulsifying and/or suspending agents. Also, certain sweetening and/or flavoring agents may be added, when needed. In an embodiment, the composition may be a formulation that allows the microorganisms, or the culture or the extract thereof to be stabilized in an acidic environment such as gastric juice. For example, the composition may be a capsule including the microorganisms, or the culture or the extract thereof therein, or a tablet in which the microorganisms, or the culture or the extract thereof are coated with a film.

A third aspect provides a food composition for preventing or ameliorating cancer including the microorganisms, or the culture or the extract thereof as an active ingredient.

The pharmaceutical composition may include a sitologically acceptable carrier. The carrier may be a stabilizer, excipient, diluent, or adjuvant. The carrier may be, for example, any and all aqueous and non-aqueous solutions, sterile solutions, solvents, buffers such as a phosphate buffered saline (PBS) solution, water, suspensions, emulsions such as oil/water emulsions, various types of wetting agents, liposomes, dispersion media and coating agents suitable for pharmaceutical administration, particularly suitable for oral administration. The use of such media and agents in food compositions is well known in the art, and compositions including such carriers may be formulated by a well-known and common method in the art.

The food may be a dairy product, a soy product, a vegetable and fruit product, or a food additive. The dairy product may be fermented milk, butter, cheese, or powdered milk. The food may be a health functional food. The health functional food may be a health functional food for preventing or improving cancer. The food may be a beverage, confectionery, diet bar, chocolate, pizza, ramen, other noodles, chewing gum, ice cream, and the like.

The food may include ingredients commonly added during food production, for example, proteins, carbohydrates, fats, nutrients, seasonings, and flavors may be included.

The carbohydrates used in food production include monosaccharides such as glucose, fructose, and the like; disaccharides such as maltose, sucrose, oligosaccharides, and the like; and polysaccharides, for example, common sugars such as dextrin, cyclodextrin, and the like, and sugar alcohols such as xylitol, sorbitol, and erythritol. Also, natural flavors and synthetic flavors such as saccharin and aspartame may be used as flavoring agents. The natural flavors may be stevia extracts such as thaumatin, rebaudioside A, and glycyrrhizin. Health functional food refers to a food that brings a specific effect on health when ingested.

In the composition, the microorganisms may be about 0.01 wt % to about 50 wt %, or about 0.1 wt % to about 20 wt %, with respect to the weight of the composition. In addition, an amount of the composition administered may be 1×10⁶ CFU/g or more, 1×10⁷ CFU/g or more, 1×10⁸ CFU/g or more, or 1×10⁹ CFU/g or more, with respect to the weight of the composition. For example, the bacterial strain may be administered in an amount of 1×10¹⁵ CFU or less, 1×10¹⁴ CFU or less, 1×10¹³ CFU or less, or 1×10¹² CFU or less. For example, the bacterial strain included in the composition may be about 1×10⁶ CFU/g to about 1×10¹⁵ CFU/g, about 1×10⁷ CFU/g to about 1×10¹⁴ CFU/g, about 1×10⁸ CFU/g to about 1×10¹³ CFU/g, about 1×10⁹ CFU/g to about 1×10¹² CFU/g, or about 1×10¹⁰ CFU/g to about 1×10¹² CFU/g.

A fourth aspect provides a method of preventing or treating cancer in a subject including administering the microorganisms, or the culture or the extract thereof in an effective amount for treating cancer.

The subject may be a mammal. The mammal may be a human or a non-human mammal. The administration may be oral administration.

The “effective amount for treating cancer” refers to an amount effective to prevent or treat cancer. The “effective amount for treating cancer” may be about 0.01 mg to about 200 mg of the above-described microorganisms, or the culture or the extract thereof per kg of body weight, or about 0.1 mg to about 400 mg of the microorganisms, or the culture or the extract thereof per kg of body weight. In addition, the “effective amount for treating cancer” administered may be 1×10⁶ CFU or more, 1×10⁷ CFU or more, 1×10⁸ CFU or more, 1×10⁹ CFU per subject. For example, the bacterial strain may be administered in an amount of 1×10¹⁵ CFU or less, 1×10¹⁴ CFU or less, 1×10¹³ CFU or less, 1×10¹² CFU or less. For example, the bacterial strain may be administered in an amount of about 1×10⁶ CFU to about 1×10¹⁵ CFU, about 1×10⁷ CFU to about 1×10¹⁴ CFU, about 1×10⁸ CFU to about 1×10¹³ CFU, about 1×10⁹ CFU to about 1×10¹² CFU, or about 1×10¹⁰ CFU to about 1×10¹² CFU. For example, a bacterial strain, which is an active ingredient, may be administered twice a day or several times a day in aliquots.

The method may include co-administering with an immune checkpoint inhibitor. The immune checkpoint inhibitor may be for administering before, concurrently with, or after the administration of the microorganisms or the culture or the extract thereof.

The immune checkpoint inhibitor may be, for example, those listed in “I. Enterococcus faecium”, but is not limited thereto.

The method may include administering in combination with a chemotherapeutic agent. The chemotherapeutic agent may be for administering before, concurrently with, or after administration of the microorganisms or the culture or the extract thereof.

The chemotherapeutic agent may be, for example, those listed in “I. Enterococcus faecium”, but is not limited thereto.

The following represents some preferred embodiments of Enterococcus faecalis LMT19-32 microorganisms with anti-tumor activity, but the present disclosure is not limited thereto:
1. Enterococcus faecalis LMT19-32 (accession number KCTC 14306BP) microorganisms having anti-tumor activity.
2. A pharmaceutical composition for preventing or treating cancer containing the microorganisms, or the culture or the extract thereof of clause 1 as an active ingredient.
3. The composition according to clause 2, wherein the cancer is a solid cancer.
4. The composition according to clause 2, which is for co-administering with an immune checkpoint inhibitor.
5. The composition according to clause 4, wherein the immune checkpoint inhibitor is a CTLA4 inhibitor, PD-1 inhibitor, or PD-L1 inhibitor.
6. A food composition for preventing or improving cancer containing the microorganisms, or the culture or the extract thereof of clause 1 as an active ingredient.

Hereinafter, the present disclosure will be described in detail by way of examples, but this is only to help understanding of the present disclosure and does not limit the scope of the present disclosure in any way.

### EXAMPLE

1. Enterococcus faecium

### Example 1: Isolation and Identification of Strains

### 1. Isolation of Strains

For isolation of Enterococcus faecium strains, infant feces, adult feces, and food samples were used. In order to isolate Enterococcus faecium strains from these samples, the samples were plated on De Man, Rogosa, and Sharpe (MRS) medium (Difco Co., USA) and cultured anaerobically at 30° C. The samples were taken aseptically, diluted with 180 ml of 0.85% NaCl solution, and homogenized with a stomacher for 5 minutes. A tube containing 9 ml of 0.85% NaCl solution was prepared by sterilizing it, and the samples were prepared by diluting in stages in the prepared tube. Thereafter, the samples were spread on an MRS plate medium and cultured at 30° C. for 2 to 3 days, and the colonies that appeared were distinguished by shape and color, and then purified again, and a total of 8 strains were selected. The selected strains were named LMT17-62, LMT17-74, LMT15-24, LMT17-25, LMT15-4, LMT17-43, LMT17-40, and LMT2-17, respectively.

### 2. Identification of Selected Strains

### 1) Analysis of Morphological Characteristics

The eight selected strains were cultured in MRS plate medium (Difco, USA), and colony morphology was observed. The colony morphologies of the selected strains are shown in Table 2 below.

TABLE 2
Morphological characteristics of selected strains

| | LMT17-62 | KCTC13225 |
|---|---|---|
| Shape | Circular | Circular |
| Size | 0.5 mm | 0.5 mm |
| Color | Cream | Cream |
| Opacity | Opaque | Opaque |
| Elevation | Convex | Convex |
| Surface | Smooth | Smooth |
| Aerobic growth | + | + |
| Anaerobic growth | + | + |

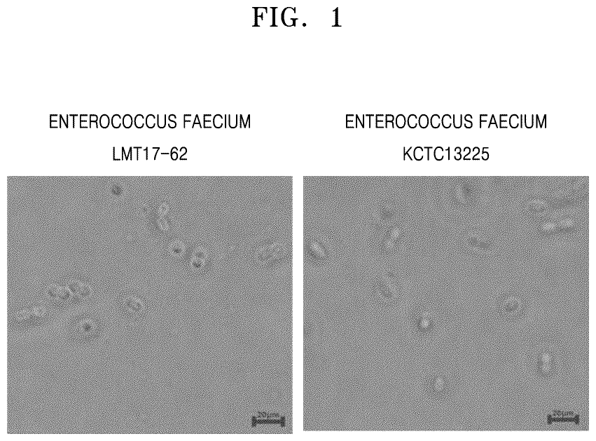

FIG. 1 shows optical microscope images of a strain Enterococcus faecium LMT17-62 among the 8 selected strains and a type strain KCTC13225. As shown in FIG. 1, the Enterococcus faecium LMT17-62 strain was confirmed to have a spherical shape typical of Enterococcus genus.

2) Analysis of 16S rDNA

In order to analyze 16S rDNA of the isolated strains, 16S rRNA genes were amplified and sequenced (Macrogen) by using universal bacterial primers (27F (SEQ ID NO: 1) and 1492R (SEQ ID NO: 2). 16S rDNA sequences of the isolated strains are shown in SEQ ID NOS: 3 to 10 (SEQ ID NO: 3: 16S rDNA sequence of LMT17-62; SEQ ID NO: 4: 16S rDNA sequence of LMT17-74; SEQ ID NO: 5: 16S rDNA sequence of LMT15-24; SEQ ID NO: 6: 16S rDNA sequence of LMT17-25; SEQ ID NO: 7: 16S rDNA sequence of LMT15-4; SEQ ID NO: 8: 16S rDNA sequence of LMT17-40; SEQ ID NO: 9: 16S rDNA sequence of LMT2-17; and SEQ ID NO: 10: 16S rDNA sequence of LMT17-43). The results were interpreted by using NCBI blast (http://www.ncbi.nlm.nih.gov/), and the isolated strains were found to belong to a species Enterococcus faecium.

The isolated strains were deposited at Korean Collection for Type Cultures (KCTC) at the Korea Research Institute of Bioscience and Biotechnology on Aug. 26, 2020, and finally named Enterococcus faecium LMT17-62 (accession number: KCTC 14284BP), Enterococcus faecium LMT17-74 (accession number: KCTC 14285BP), Enterococcus faecium LMT15-24 (accession number: KCTC 14289BP), Enterococcus faecium LMT17-25 (accession number: KCTC 14288BP), Enterococcus faecium LMT15-4 (accession number: KCTC 14290BP), Enterococcus faecium LMT17-43 (accession number: KCTC 14286BP), Enterococcus faecium LMT17-40 (accession number: KCTC 14287BP) and Enterococcus faecium LMT2-17 (accession number: KCTC 14291BP), respectively.

### Example 2: Evaluation of Anti-Tumor Efficacy of Selected Enterococcus faecium Strains

1. Induction of Tumor Models in Mice and Administration of Enterococcus faecium Strains

C57BL/6 mice (male, 20 g to 22 g) used as mouse tumor induction models were purchased from Orient Bio Co., Ltd. and were acclimated to the environment for 1 week before the start of the experiment. 2.5×10⁵ MC38 cells derived from colorectal cancer of C57BL/6 mice were injected into a subcutaneous tissue of the mouse back, and after 1 week of tumor injection, groups were separated based on tumor sizes (50 mm³ to 70 mm³). After the group separation, 1.0×10⁹ CFU of Enterococcus faecium strain per mouse was orally administered every other day for 2 weeks by using a sonde. For a positive control group, 10 mg of anti-PD1 antibodies (clone number: RMP1-14) per 1 kg of mouse weight was intraperitoneally administered twice a week. For a negative control group, phosphate buffered saline (PBS) was orally administered.

Tumor sizes were measured up to 21 days after the MC38 tumor cell line was injected into the mice, and then the mice were sacrificed by using carbon dioxide, and the tumor and immune organs were extracted to perform an analysis of tumor-infiltrating immune cells.

2. Evaluation of Anti-Tumor Efficacy According to Administration of Enterococcus faecium Strains

To investigate tumor suppression efficacy of the Enterococcus faecium strains, tumor suppression experiments were conducted on 8 selected microbial strains.

Sizes of the tumor were measured twice a week from day 7 after the injection of the tumor cell line. In order to accurately measure sizes of the tumor, the long axis and the short axis of the tumor were measured by using a Vernier caliper, and the tumor sizes were calculated according to the equation below:

$$Tumor size = {( long axis \times ( short axis )}^{2} ) / 2.$$

The tumor growth inhibition rates (%) were calculated according to the equation below:

$$Tumor growth inhibition rate ( \% ) = ( 1 - Vt / Vc ) \times 100 ,$$

wherein in the equation, Vc is a mean tumor size in the negative control group when evaluating a tumor size, and
Vt is a mean tumor size in the experimental group when evaluating a tumor size.

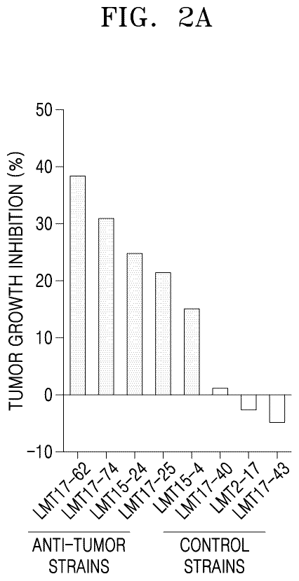

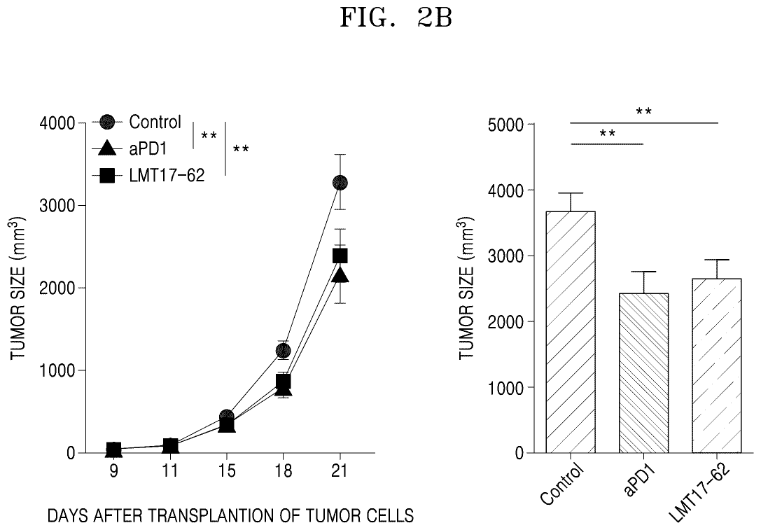

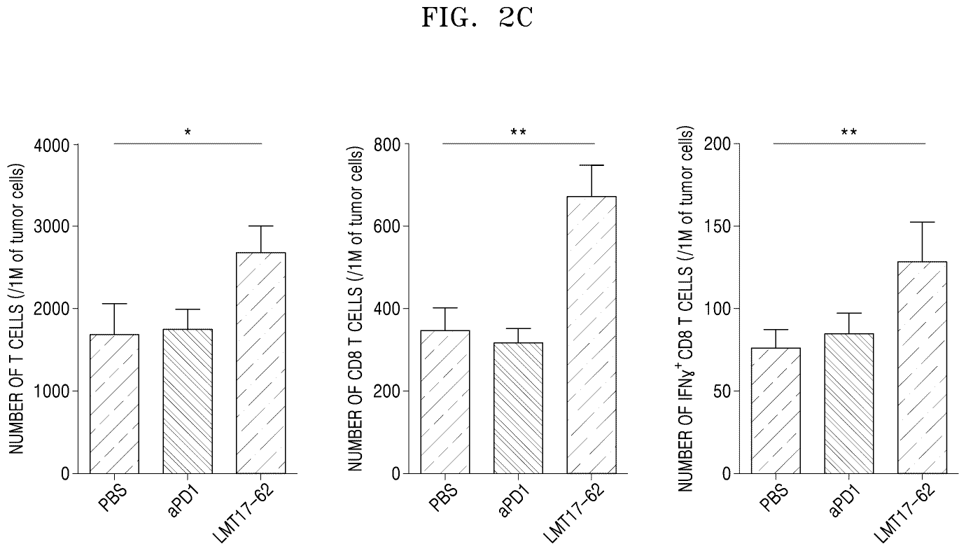

The results are shown in FIG. 2A. As shown in FIG. 2A, Enterococcus faecium strains LMT17-62, LMT17-74, LMT15-24, LMT17-25, and LMT15-4 showed tumor growth inhibition rates of 39%, 31%, 25%, 22%, and 15%, respectively. On the other hand, Enterococcus faecium strains LMT17-40, LMT2-17, and LMT17-43 did not show anti-tumor efficacy with tumor growth inhibition rates of 1%,-3%, and-5% or less, respectively. Therefore, it was confirmed that even when strains belong to the same species, there are differences in anti-tumor efficacy depending on the strain.

FIG. 2B shows tumor sizes of up to 21 days when the experiment was completed after the injection of the tumor cell line in the group administered with Enterococcus faecium LMT17-62 strain. As shown in FIG. 2B, it was confirmed that tumor growth was suppressed statistically significantly in the group administered with Enterococcus faecium LMT17-62, and the positive control group administered with anti-PD1 antibodies, compared to the negative control group.

### 3. Analysis of Tumor-Infiltrating Immune Cells and Evaluation of Functionality of Immune Cells

CD8 T cells, which are tumor-infiltrating immune cells, are important markers of anti-tumor responses, and interferon gamma secreted by CD8 T cells is a functional cytokine that indicates activity of immune cells. In order to confirm whether the anti-tumor efficacy of Enterococcus faecium was due to an increase of the above factors, tumors were excised and analyzed after the animal experiments were completed. The excised tumors were separated into single cells by using RPMI1640 medium containing 50 μg/ml of liberase and 40 μg/ml of DNase I, and a cell strainer. The isolated cells were stimulated for 4 hours with 50 ng/ml of phorbol 12-myristate 13-acetate (PMA) and 500 ng/ml of ionomycin in RPMI1640 medium containing 10% fetal bovine serum. After the stimulation, the cells were stained with the antibodies in Table 3 and analyzed by using CANTO II flow cytometry equipment for immune cell analysis and confirmation of interferon gamma production.

TABLE 3
List of antibodies for flow cytometry

| Description | Target | Color |
|---|---|---|
| Surface marker | CD45 | PE-Cy7 |
| | TCRβ | APC-Cy7 |
| | CD4 | PerCP-Cy5.5 |
| | CD8 | Pacific blue |
| | PD1 | APC |
| Intracellular staining | IFN-γ | FITC |

FIG. 2C shows a total number of tumor-infiltrating T cells, CD8 T cells, and interferon gamma-producing CD8 T cells. Tumor-infiltrating T cells and CD8 T cells were significantly increased in a group treated with a LMT17-62 strain compared to a group treated with PBS. In addition, it was confirmed that production of interferon gamma, a CD8 T cell activator, was also significantly increased in the group treated with LMT17-62 compared to the PBS control group.

### Example 3: Analysis of Physiological Activity Characteristics of Anti-Tumor-Positive Enterococcus faecium Strains

1. Evaluation of Growth Curve and Metabolite Production Ability of Anti-Tumor-Positive Enterococcus faecium Strains

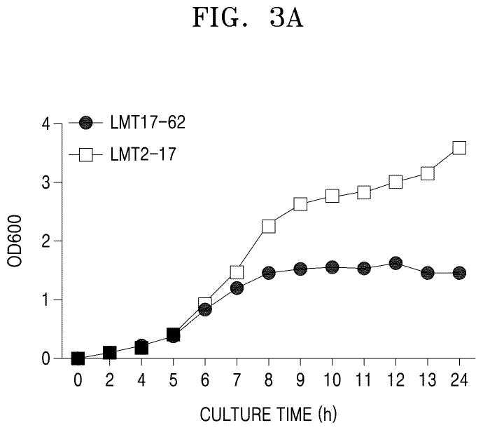

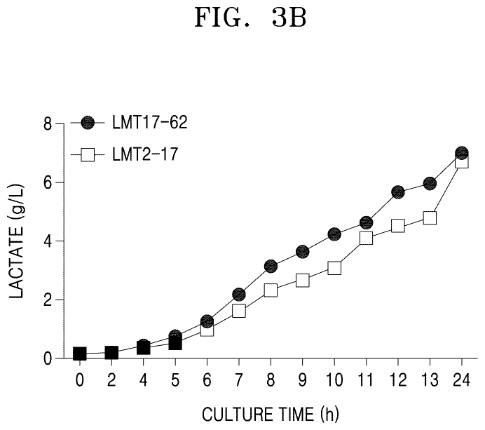

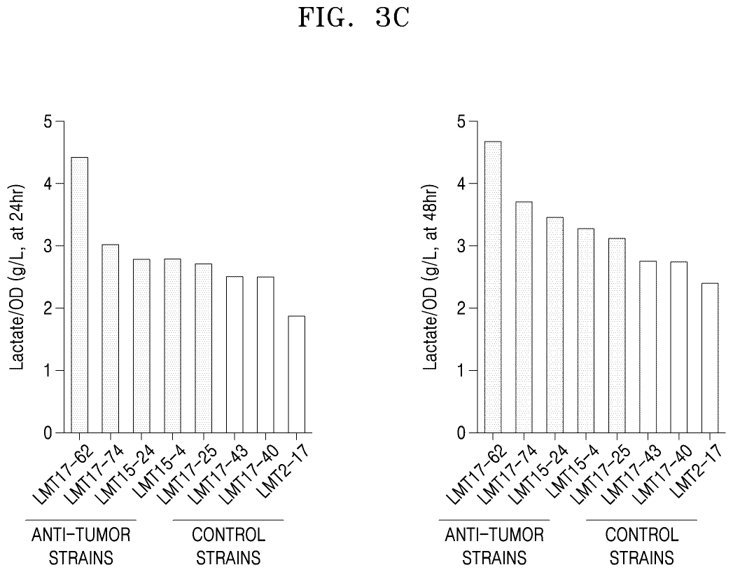

In order to examine growth patterns of the selected Enterococcus faecium strains, colonies of anti-tumor-positive strains and anti-tumor-negative strains were cultured in MRS liquid medium for 24 hours, and then the strains were inoculated into 50 ml or 250 ml Erlenmeyer flasks, such that an initial optical density value of each strain at a wavelength of 600 nm (OD₆₀₀) was identical. Then, in order to measure growth curves of the strains, OD₆₀₀ values of the strains were measured every hour by using a spectrophotometer Ultrospec 7000 (biochrom, UK). The results are shown in FIG. 3A. As shown in FIG. 3A, the LMT17-62 strain with anti-tumor efficacy stopped growing after 8 hours of culture, but the anti-tumor negative LMT2-17 strain continued to grow. Amounts of lactate, a metabolite of the strains present in the culture medium, were confirmed by using a biochemical analyzer YSI2900 (Xylem, USA).

The results are shown in FIG. 3B. As shown in FIG. 3B, production of lactate, which is a metabolite, increased as culturing of both the LMT17-62 strain having anti-tumor efficacy and the anti-tumor-negative LMT2-17 strain was continued. FIG. 3C shows abilities to produce lactate, a sugar metabolite, per OD₆₀₀. As shown in FIG. 3C, the group of Enterococcus faecium strains with anti-tumor efficacy (LMT17-62, LMT17-74, LMT15-24, LMT15-4, and LMT17-25) showed higher capacity to produce lactate per OD₆₀₀ then the group of anti-tumor-negative Enterococcus faecium strains (LMT17-43, LMT17-40, and LMT2-17). This was shown more pronounced as the strains were continued to be cultured from 24 hours to 48 hours. That is, when cultured for 48 hours, the anti-tumor-negative strains showed lactate production per OD₆₀₀ of less than 3 g/L, whereas the anti-tumor-positive strains consistently showed lactate production per OD₆₀₀ of 3 g/L or more, and the higher the lactate production ability per OD₆₀₀, the higher the anti-tumor activity.

2. Evaluation of Enzyme Activity of Anti-Tumor-Positive Enterococcus faecium Strains

In order to investigate biochemical characteristics of the selected Enterococcus faecium strains, enzyme activities were evaluated by using a Rapid ID 32A kit (BioMetrieux, France), and the results are shown in Table 4 below. Unlike the group of anti-tumor-negative strains (LMT17-43, LMT17-40, and LMT2-17), the group of Enterococcus faecium strains with anti-tumor efficacy (LMT17-62, LMT17-74, LMT15-24, and LMT17-25) had ß-galactosidase enzyme activity.

TABLE 4

| | | Anti-tumor strain | | | | | Control strain | | |
|---|---|---|---|---|---|---|---|---|---|
| Number | Enzyme | No. 1 | No. 2 | No. 3 | No. 4 | No. 5 | No. 1 | No. 2 | No. 3 |
| 1 | Urease | − | − | − | − | − | − | − | − |
| 2 | Arginine Dihydrolase | + | + | + | + | + | + | + | + |
| 3 | α-galactosidase | − | − | − | − | − | + | + | + |
| 4 | β-galactosidase | + | + | + | + | − | − | − | − |
| 5 | β-galactosidase | − | − | − | − | − | − | − | − |
| | 6-phosphate | | | | | | | | |
| 6 | α-glucosidase | − | − | − | − | − | − | − | − |
| 7 | β-glucosidase | − | − | − | − | − | − | − | − |
| 8 | α-arabinosidase | − | − | − | − | − | − | − | − |
| 9 | β-glucuronidase | − | − | − | − | − | − | − | − |
| 10 | N-acetyl-β | − | − | − | − | − | − | − | − |
| | glucosaminidase | | | | | | | | |
| 11 | Mannose fermentation | + | + | + | + | + | + | + | + |
| 12 | Raffinose | + | − | − | − | − | + | + | + |
| | fermentation | | | | | | | | |
| 13 | Glutamic acid | − | − | − | − | − | − | − | − |
| | decarboxylase | | | | | | | | |
| 14 | α-fucosidase | − | − | − | − | − | − | − | − |
| 15 | Nitrate reduction | − | − | − | − | − | − | − | − |
| 16 | Indole production | + | + | + | + | + | + | + | + |
| 17 | Alkaline phosphatase | − | − | − | − | − | − | − | − |
| 18 | Arginine arylamidase | + | + | + | + | + | + | + | + |
| 19 | Proline arylamidase | − | − | − | − | − | − | − | − |
| 20 | Leucyl glycine | − | − | − | − | − | − | − | − |
| | arylamidase | | | | | | | | |
| 21 | Phenylalanine | + | + | + | + | + | + | + | + |
| | arylamidase | | | | | | | | |
| 22 | Leucine arylamidase | − | + | + | + | + | + | + | + |
| 23 | Pyroglutamic acid | + | + | + | + | + | + | + | + |
| | arylamidase | | | | | | | | |
| 24 | Tyrosine arylamidase | + | + | + | + | + | + | + | + |
| 25 | Alanine arylamidase | − | − | − | − | − | − | − | − |
| 26 | Glysine arylamidase | − | + | + | + | + | + | + | + |
| 27 | Histidine arylamidase | − | + | + | + | + | + | + | + |
| 28 | Glutamyl glutamic | − | − | − | − | − | − | − | − |
| | acid arylamidase | | | | | | | | |
| 29 | Serine arylamidase | − | + | + | + | + | + | + | + |

In Table 4, the anti-tumor strains No.1, No.2, No.3, No.4, and No.5 are respectively LMT17-62, LMT17-74, LMT15-24, LMT17-25, and LMT15-4, in this order, and control strains No.1, No.2, and No.3 respectively represent LMT17-43, LMT17-40, and LMT2-17, in this order.

3. Evaluation of Sugar Fermentation Characteristics of Anti-Tumor-Positive Enterococcus faecium Strains

In order to analyze sugar metabolism characteristics of the selected Enterococcus faecium strains, evaluation was performed by using an API 50 CHL kit (BioMetrieux, France), according to manufacturer's instructions. The results are shown in Table 5. The group of Enterococcus faecium strains with anti-tumor efficacy (LMT17-62, LMT17-74, and LMT15-24) had D-sorbitol and D-tagatose sugar fermentation characteristics. In addition, in the case of Enterococcus faecium strains (LMT17-62, and LMT17-74) with anti-tumor efficacy, the strains had methyl-αD-mannopyranoside sugar fermentation characteristics.

TABLE 5

| | | Anti-tumor strain | | | | | Control strain | | |
|---|---|---|---|---|---|---|---|---|---|
| Number | Carbohydrate | No. 1 | No. 2 | No. 3 | No. 4 | No. 5 | No. 1 | No. 2 | No. 3 |
| 1 | Glycerol | − | + | − | − | − | − | − | − |
| 2 | Erythritol | − | − | − | − | − | − | − | − |
| 3 | D-arabinose | − | − | − | − | − | − | − | − |
| 4 | L-arabinose | + | + | + | + | + | + | + | + |
| 5 | D-ribose | + | + | + | + | + | + | + | + |
| 6 | D-xylose | − | − | − | − | − | − | − | − |
| 7 | L-xylose | − | − | − | − | − | − | − | − |
| 8 | D-adonitol | − | − | − | − | − | − | − | − |
| 9 | Methyl-βD- | − | − | − | − | − | − | − | − |
| | xylopyranoside | | | | | | | | |
| 10 | D-galactose | + | + | + | + | + | + | + | + |
| 11 | D-glucose | + | + | + | + | + | + | + | + |
| 12 | D-fructose | + | + | + | + | + | + | + | + |
| 13 | D-mannose | + | + | + | + | + | + | + | + |
| 14 | L-sorbose | − | − | − | − | − | − | − | − |
| 15 | L-rhamnose | − | − | − | + | − | − | + | − |
| 16 | Dulcitol | + | − | − | − | − | − | − | − |
| 17 | Inositol | − | − | − | − | − | − | − | − |
| 18 | D-mannitol | + | + | + | + | + | + | + | + |
| 19 | D-sorbitol | + | + | + | − | − | − | − | − |
| 20 | Methyl-αD- | + | + | − | − | − | − | − | − |
| | mannopyranoside | | | | | | | | |
| 21 | Methyl-αD- | − | − | − | − | − | − | − | − |
| | glucopyranoside | | | | | | | | |
| 22 | N-acetylglucosamine | + | + | + | + | + | + | + | + |
| 23 | Amygdalin | + | + | + | + | − | + | + | + |
| 24 | Arbutin | + | + | + | + | + | + | + | + |
| 25 | Aesculin | + | + | + | + | + | + | + | + |
| 26 | Salicin | + | + | + | + | + | + | + | + |
| 27 | D-cellobiose | + | + | + | + | + | + | + | + |
| 28 | D-maltose | + | + | + | + | + | + | + | + |
| 29 | D-lactose | + | + | + | + | + | + | + | + |
| 30 | D-melibiose | + | + | + | − | + | + | + | + |
| 31 | D-saccharose | + | + | + | + | + | + | + | + |
| 32 | D-trehalose | + | + | + | + | + | + | + | + |
| 33 | Inulin | − | − | − | − | − | − | − | − |
| 34 | D-melezitose | − | + | − | − | − | − | − | − |
| 35 | D-raffinose | + | − | + | − | − | + | + | + |
| 36 | Amidone | − | − | − | − | − | − | − | − |
| 37 | Glycogen | − | − | − | − | − | − | − | − |
| 38 | Xylitol | − | − | | − | − | − | − | − |
| 39 | Gentiobios | + | + | + | + | + | + | + | + |
| 40 | D-turanose | − | − | − | − | − | − | − | − |
| 41 | D-lyxose | − | − | − | − | − | − | − | − |
| 42 | D-tagatose | + | + | + | − | − | − | − | − |
| 43 | D-fucose | − | − | − | − | − | − | − | − |
| 44 | L-fucose | − | − | − | − | − | − | − | − |
| 45 | D-arabitol | − | − | − | − | − | − | − | − |
| 46 | L-arabitol | − | − | − | − | − | − | − | − |
| 47 | Potassium gluconate | + | + | + | + | − | − | + | − |
| 48 | Potassium 2- | − | − | − | − | − | − | − | − |
| | ketogluconate | | | | | | | | |
| 49 | Potassium 5- | − | − | − | − | − | − | − | − |
| | ketogluconate | | | | | | | | |

In Table 5, the anti-tumor strains No.1, No.2, No.3, No.4, and No.5 are respectively LMT17-62, LMT17-74, LMT15-24, LMT17-25, and LMT15-4, in this order, and control strains No.1, No.2, and No.3 respectively represent LMT17-43, LMT17-40, and LMT2-17, in this order.

4. Evaluation of Stability of Anti-Tumor-Positive Enterococcus faecium Strains

### (1) Investigation of Acid Resistance

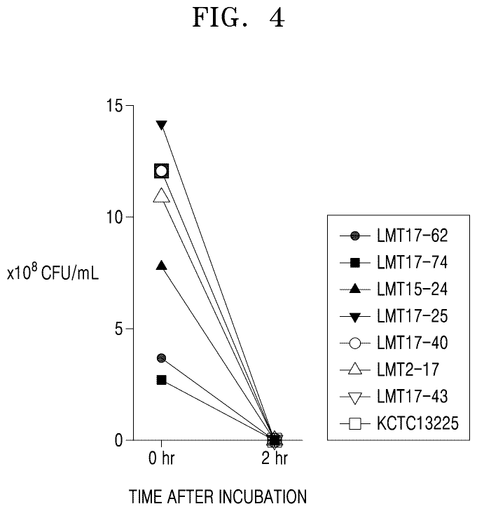

In order to evaluate acid resistance of Enterococcus faecium strains, an experiment was conducted in the following manner. The selected strains were inoculated into sterilized MRS liquid medium and then cultured at 37° C. for 16 hours. Then, after inoculating the strains into sterilized MRS liquid medium, in which pH was adjusted to 2.5 by using HCl, the cells were incubated at 37° C. for 2 hours, and numbers of live bacteria were confirmed. Live bacteria before inoculation and live bacteria at 2 hours after the inoculation were spread on MRS plate medium and then cultured, and numbers of colonies thereof were counted and compared, and the results are shown in FIG. 4.

As shown in FIG. 4, since the anti-tumor-positive Enterococcus faecium strain was shown to have somewhat low acid resistance, it may be preferable to use a capsule or a coating agent during formulation.

### (2) Examination of Bile Resistance

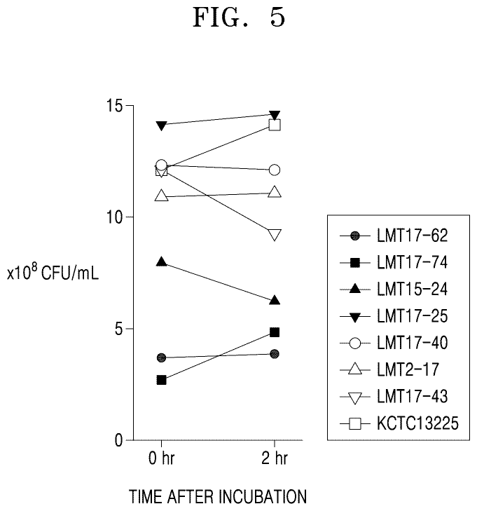

In order to examine bile resistance of Enterococcus faecium strains, an experiment was conducted in the following manner. The selected strains were inoculated into sterilized MRS liquid medium and then cultured at 37° C. for 16 hours. Considering that a concentration of bile salts in the intestinal tract is around 0.1%, only the bacterial cells were inoculated into MRS liquid medium containing 0.3% of bile salts (Sigma, USA) to 108 CFU/ml to 109 CFU/ml, and cultured at 37° C. for 2 hours, then numbers of live bacteria were identified. Live bacteria before inoculation and live bacteria at 2 hours after the inoculation were spread on MRS plate medium and then cultured to count numbers of colonies thereof. The results are shown in FIG. 5.

As shown in FIG. 5, since the anti-tumor-positive Enterococcus faecium strains maintained an appropriate number of bacteria even at 0.3%, which is higher than 0.1%, which is similar to the actual concentration in the intestine, it may be determined that the selected anti-tumor-positive Enterococcus faecium strains may survive sufficiently in the intestines of humans or animals.

### (3) Investigation of Intestinal Anchorage

In order to evaluate intestinal cell anchorage of the anti-tumor-positive Enterococcus faecium strains, a Caco-2 cell line (KCLB 30037.1), which is human epithelial colorectal adenocarcinoma cells purchased from the Korea Cell Line Bank, was used. Caco-2 cells were divided in Dulbecco's modified Eagle's medium (DMEM; Gibco, USA) containing 10% fetal bovine serum (FBS) (Gibco, USA) under conditions of 5% CO₂ and 37° C., to be 7×10⁴ cells/100 μl, and cultured to form a monolayer of cells in a 96-well plate (Corning, USA).

On the other hand, the selected anti-tumor-positive Enterococcus faecium strains cultured in MRS liquid medium were washed with phosphate buffered saline (PBS), suspended in antibiotic-free DMEM medium, and added to Caco-2 cells that formed a monolayer of cells, so that amounts of the Enterococcus faecium strains would be 1×10⁷ CFU, and the selected anti-tumor-positive Enterococcus faecium strains were cultured for 2 hours under conditions of 5% CO₂ and 37° C. In order to remove cells that failed to adhere to the Caco-2 cells, the cells were washed 5 times with PBS, and the adhered cells were detached with 100 μl of 0.1% Triton x-100 and then spread on MRS solid medium. After incubation at 37° C. for 24 hours, numbers of colonies on the plate medium were counted to examine intestinal anchorage of the Enterococcus faecium strains.

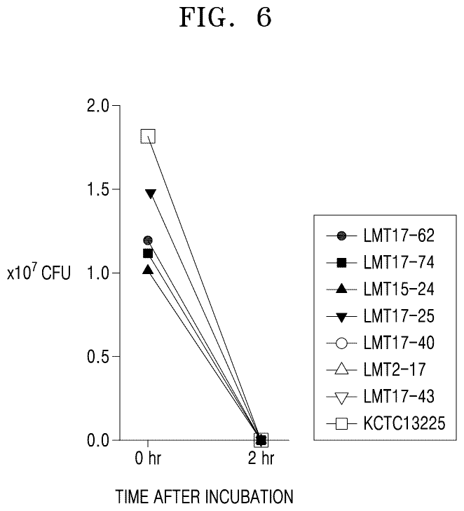

FIG. 6 shows numbers of the selected anti-tumor-positive Enterococcus faecium strains attached to intestinal epithelial cells. As shown in FIG. 6, it was confirmed that LMT17-62, LMT17-74, LMT15-24, and LMT17-25 had intestinal anchorage on intestinal epithelial cells (Caco-2) of 0.31%, 1.55%, 0.46%, and 0.48%, respectively.

### II. Enterococcus faecalis LMT19-32

### Example 1: Isolation and Identification of Enterococcus faecalis LMT19-32 Strain

1. Isolation of Enterococcus faecalis Strains

Enterococcus faecalis strains were isolated from adult fecal samples. First, the samples were spread on MRS medium (Difco Co., USA) and cultured anaerobically at 30° C. As a pretreatment method of the sample, the sample was taken aseptically, diluted with 180 ml of 0.85% NaCl solution, and the fecal solution was homogenized by using a stomacher for 5 minutes. A fecal sample was prepared by diluting the homogenized sample in stages in a tube containing 9 ml of a sterile 0.85% NaCl solution. The sample was spread on MRS plate medium (Difco, USA) and cultured at 37° C. for 2 days to 3 days, and the colonies that appeared were distinguished according to shape and color, and then purified and separated again.

2. Identification of Enterococcus faecalis Strains

### (1) Analysis of Morphological Characteristics

A selected strain was cultured in MRS plate medium (Difco, USA), and colony morphology was observed. Colony morphology of the selected Enterococcus faecalis strain and the Enterococcus faecalis type strain KCTC3206, on the MRS plate medium is shown in Table 6 below.

TABLE 6

| | LMT19-32 | KCTC3206 |
|---|---|---|
| Shape | Circular | Circular |
| Size | 0.8 mm | 0.8 mm |
| Color | Cream | Cream |
| Transparency | Opaque | Opaque |
| Elevation | Convex | Convex |
| Surface | Smooth | Smooth |
| Aerobic growth | + | + |
| Anaerobic growth | + | + |

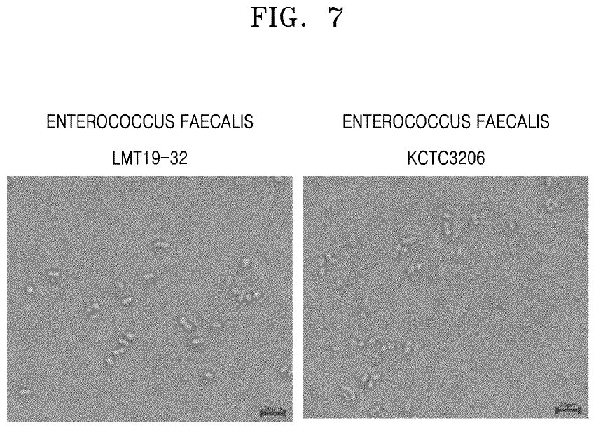

FIG. 7 shows representative optical microscope images of the selected strain Enterococcus faecalis LMT19-32 and the type strain KCTC3206. As shown in FIG. 7, the LMT19-32 strain was coccus and was similar in morphology to a typical Enterococcus genus.

(2) Analysis of 16S rDNA

16S rRNA genes of the isolated LMT19-32 strain were amplified, and a nucleotide sequence of the amplified 16S rRNA genes was analyzed. The amplification was performed by PCR by using genomic DNA of the LMT19-32 strain as a template and an oligonucleotide of SEQ ID NO: 11 and an oligonucleotide of SEQ ID NO: 12 (Macrogen) as a primer set. The nucleotide sequence of 16S rDNA of the isolated LMT19-32 strain is shown in SEQ ID NO: 13. The nucleotide sequence of the identified 16S rDNA was compared with the nucleotide sequence of the known 16S rDNA by using NCBI blast (http://www.ncbi.nlm.nih.gov/). As a result, the 16S rDNA of LMT19-32 had 100% sequence identity with that of the Enterococcus faecalis species. In addition, as a result of phylogenetic tree analysis, LMT19-32 was the same as the Enterococcus faecalis species. As a result, LMT19-32 strain was identified as a new strain belonging to a Enterococcus faecalis species.

The present inventors named LMT19-32 lactic acid bacteria as “Enterococcus faecalis LMT19-32” (accession number: KCTC14306BP) and deposited the same to the Korean Collection for Type Cultures (KCTC) at the Korea Research Institute of Bioscience and Biotechnology, on Sep. 9, 2020.

### Example 2: Analysis of Physiological Activity Characteristics of Enterococcus faecalis LMT19-32 Strain

1. Sugar Fermentation Characteristics of Strain Enterococcus faecalis LMT19-32

Sugar metabolism characteristics of the selected LMT19-32 strain were confirmed by using an API 50 CHL kit (BioMetrieux, France) according to the manufacturer's experimental method. Table 7 shows sugar fermentation characteristics of the identified LMT19-32 strain.

TABLE 7

| | | LMT19-32 | |
|---|---|---|---|
| Number | Carbohydrate | 24 hours | 48 hours |
| 1 | Glycerol | + | + |
| 2 | Erythritol | − | − |
| 3 | D-arabinose | − | − |
| 4 | L-arabinose | − | − |
| 5 | D-ribose | + | + |
| 6 | D-xylose | − | − |
| 7 | L-xylose | − | − |
| 8 | D-adonitol | − | − |
| 9 | Methyl-bD-xylopyranoside | − | − |
| 10 | D-galactose | + | + |
| 11 | D-glucose | + | + |
| 12 | D-fructose | + | + |
| 13 | D-mannose | + | + |
| 14 | L-sorbose | − | − |
| 15 | L-rhamnose | − | − |
| 16 | Dulcitol | − | − |
| 17 | Inositol | − | + |
| 18 | Mannitol | + | + |
| 19 | D-sorbitol | + | + |
| 20 | Methyl-αD-mannopyranoside | − | − |
| 21 | Methyl-αD-glucopyranoside | − | − |
| 22 | N-acetylglucosamine | + | + |
| 23 | Amygdalin | + | + |
| 24 | Arbutin | + | + |
| 25 | Aesculin | + | + |
| 26 | Salicin | + | + |
| 27 | D-cellobiose | + | + |
| 28 | D-maltose | + | + |
| 29 | D-lactose | + | + |
| 30 | D-melibiose | − | − |
| 31 | D-saccharose | + | + |
| 32 | D-trehalose | + | + |
| 33 | Inulin | − | − |
| 34 | D-melezitose | + | + |
| 35 | D-raffinose | − | − |
| 36 | Amidone | − | − |
| 37 | Glycogen | − | − |
| 38 | Xylitol | − | − |
| 39 | Gentiobios | + | + |
| 40 | D-turanose | − | − |
| 41 | D-lyxose | − | − |
| 42 | D-tagatose | + | + |
| 43 | D-fucose | − | − |
| 44 | L-fucose | − | − |
| 45 | D-arabitol | − | − |
| 46 | L-arabitol | − | − |
| 47 | Potassium gluconate | + | + |
| 48 | Potassium 2-ketogluconate | − | − |
| 49 | Potassium 5-ketogluconate | − | − |

2. Evaluation of Stability of Enterococcus faecalis LMT19-32 Strain

In order to evaluate acid resistance of Enterococcus faecalis LMT19-32 strain, an experiment was conducted in the following manner. The LMT19-32 strain was inoculated into sterilized MRS liquid medium and then cultured at 37° C. for 16 hours. Then, 1% of the strain was inoculated into a sterilized MRS liquid medium adjusted to pH 2.5 with HCl, and cultured at 37° C. for 2 hours. Samples immediately after the strain inoculation and after 2 hours of incubation were collected, diluted in MRS liquid medium, spread on MRS plate medium, incubated at 37° C. for 24 hours, and then the number of colonies on the plate medium was counted to measure the number of bacteria.

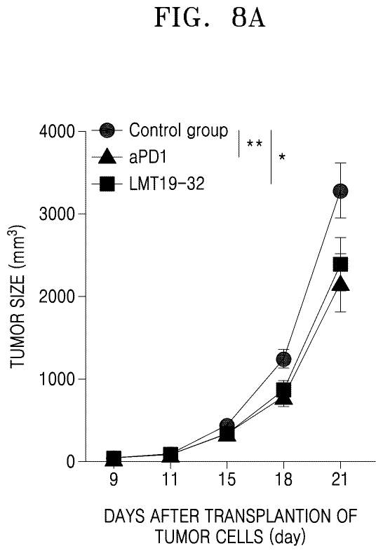

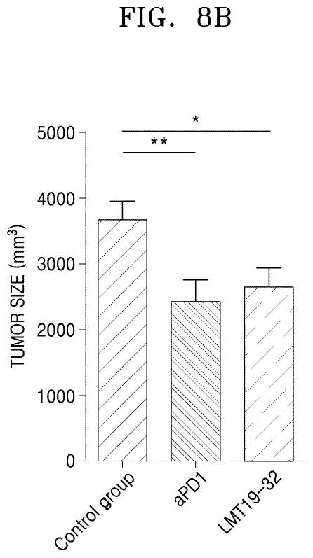

As shown in FIG. 8, since the Enterococcus faecalis LMT19-32 strain was shown to have somewhat low acid resistance, it may be preferable to use a capsule or a coating agent during formulation.

TABLE 8

| | LMT19-32 | KCTC3206 |
|---|---|---|
| MRS (pH 6.8) (cell number/plate) | 3.1 × 10⁹ | 1.8 × 10⁹ |
| MRS (pH 2.5) (cell number/plate) | 3.0 × 10¹ | 8.5 × 10⁴ |
| Survival rate (%) | 0.000001 | 0.004670 |

### (2) Examination of Bile Resistance

In order to confirm effects of bile acid on the Enterococcus faecalis LMT19-32 strain, an experiment was conducted in the following manner. The selected strain were inoculated into sterilized MRS liquid medium and then cultured at 37° C. for 24 hours. Considering that a concentration of bile salts in the intestine is around 0.1%, 1% of the strain was inoculated into MRS liquid medium containing 0.3% of bile salts (Sigma, USA) and cultured at 37° C. for 2 hours. Samples immediately after the strain inoculation and after 2 hours of incubation were collected, diluted in MRS liquid medium, spread on MRS plate medium, incubated at 37° C. for 24 hours, and then the number of colonies on the plate medium was counted to measure the number of viable cells of the strain. As a control group, culturing was performed in the same manner in MRS liquid medium without 0.3% bile salts, and the number of viable cells of the strain was counted. Table 9 shows results of measuring bile salt resistance. As shown in Table 9, the LMT19-32 strain maintained a survival rate of 93% at 0.3%, which is higher than 0.1%, which is similar to the actual concentration in the intestine, and therefore, LMT19-32 strain may be determined to survive in the intestine of humans or animals.

TABLE 9

| | LMT19-32 | KCTC3206 |
|---|---|---|
| Control group (cell number/plate) | 3.1 × 10⁹ | 1.8 × 10⁹ |
| 0.3% bile salt (cell number/plate) | 2.9 × 10⁹ | 1.4 × 10⁹ |
| Survival rate (%) | 93.0 | 80.2 |

### (3) Investigation of Intestinal Anchorage

In order to evaluate intestinal cell anchorage of the Enterococcus faecalis LMT19-32 strain, a Caco-2 cell line (KCLB 30037.1), which is human epithelial colorectal adenocarcinoma cells purchased from the Korea Cell Line Bank, was used. Caco-2 cells were divided in Dulbecco's modified Eagle's medium (DMEM; Gibco, USA) containing 10% fetal bovine serum (FBS) (Gibco, USA) under conditions of 5% CO₂ and 37° C., to be 7×10⁴ cells/100 μl, and cultured to form a monolayer of cells in a 96-well plate (Corning, USA).

On the other hand, the LMT19-32 strain cultured in MRS liquid medium was washed with PBS, then suspended in DMEM medium to which antibiotics are not added, and the LMT19-32 strain was added to Caco-2 cells forming a monolayer of cells to 1×10⁷ CFU, and was incubated for 2 hours under conditions of 5% CO₂ and 37° C. In order to remove cells that failed to adhere to the Caco-2 cells, the cells were washed 5 times with PBS, and the adhered cells were detached with 100 μl of 0.1% Triton x-100 and then spread on MRS solid medium. After incubation at 37° C. for 24 hours, numbers of colonies on the plate medium were counted to examine intestinal anchorage of the LMT19-32 strain.

Table 10 is a diagram showing a number of colonies attached to intestinal epithelial cells of Enterococcus faecalis LMT19-32 strain. As shown in Table 10, it was confirmed that both the novel Enterococcus faecalis LMT19-32 strain of the present disclosure and the comparative strain Enterococcus faecalis KCTC3206 had the ability to anchor to Caco-2, which is intestinal epithelial cells of about 1%.

TABLE 10

| | LMT19-32 | KCTC3206 |
|---|---|---|
| Number of treated strains (10⁷ CFU) | 1.4 | 1.5 |
| Number of anchored strains (10⁴ CFU) | 10.4 | 19.4 |
| Anchorage rate (%) | 0.75 | 1.26 |

### Example 3: Analysis of Anti-Tumor Efficacy of Enterococcus faecalis LMT19-32 Strain 1. Induction of Tumor Models in Mice and Administration of Enterococcus faecalis LMT19-32 Strain

C57BL/6 mice (male, 20 g to 22 g) used as mouse tumor induction models were purchased from Orient Bio Co., Ltd. and were acclimated to the environment for 1 week before the experiment began. 2.5×10⁵ MC38 cells derived from colorectal cancer of C57BL/6 mice were injected into a subcutaneous tissue of the mouse back, and after 1 week of tumor injection, groups were separated based on tumor sizes (50 mm³ to 70 mm³). After the group separation, PBS containing 1.0×10⁹ CFU of the Enterococcus faecalis LMT19-32 strain per mouse was orally administered every other day for 2 weeks by using a sonde. For a positive control group, 10 mg of anti-PD1 antibodies (clone number: RMP1-14, manufacturer: Bioxcell, product name: InvivoMAb anti-mouse PD-1) per 1 kg of mouse weight was intraperitoneally administered twice a week. As a negative control group, PBS was orally administered. The experimental groups were configured as shown in Table 11, with a total of 3 groups of 10 animals in each group.

TABLE 11

| | |
|---|---|
| Group 1 | PBS control group |
| Group 2 | Anti-PD1 antibodies-treated group |
| Group 3 | Enterococcus faecalis LMT19-32-treated group |

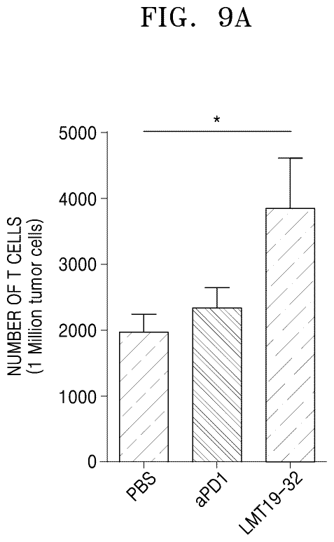

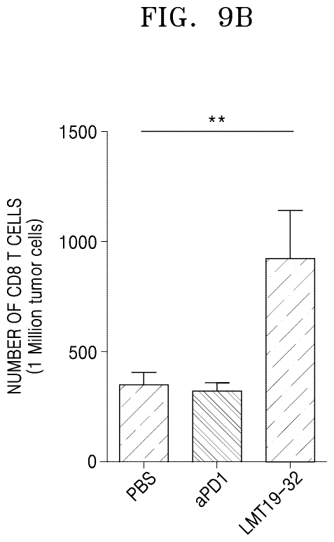

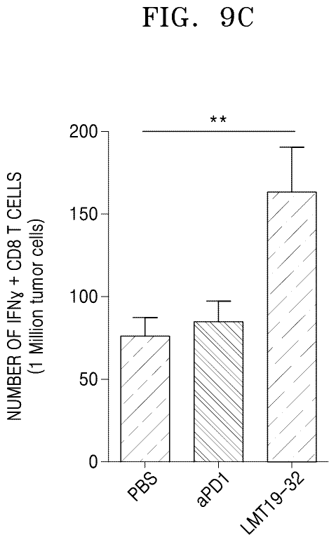

Tumor sizes were measured up to 21 days after the MC38 tumor cell line was injected into the mice, and then the mice were sacrificed by using carbon dioxide, and the tumor was extracted to perform an analysis of tumor-infiltrating immune cells. The analysis results are shown in FIGS. 9A, 9B and 9C. FIGS. 9A, 9B, and 9C show results of analyzing tumor-infiltrating T cells, CD8 T cells, and IFN_{γ+} CD8 T cells, which express IFNγ, after the administration of the Enterococcus faecalis LMT19-32 strain to mice with tumor. The MC38 tumor cell line is mouse colon adenocarcinoma cells induced by subcutaneous injection of dimethylhydrazine into C57BL/6 mice. Since the mouse tumor-induced models used in this example had MC38 tumor cells transplanted into the subcutaneous tissue, it is indicated that an effect of the orally administered strain inhibits cancer growth in tissues other than tissues directly contacted by the strain, such as subcutaneous tissue, and that the effect of the bacteria may not only act on cancers associated with the gastrointestinal tract or intestine, which is a tissue in direct contact with the strain, but also on various solid carcinomas.

2. Evaluation of Anti-Tumor Efficacy According to Administration of Enterococcus faecalis LMT19-32 Strain

FIGS. 8A and 8B are diagrams showing results of measuring tumor sizes after administering the Enterococcus faecalis LMT19-32 strain to mice with tumor. The administration was performed according to Section 1. Specifically, sizes of the tumor were measured twice a week from day 7 after the injection of the tumor cell line to day 21. In order to accurately measure the sizes of the tumor, the long axis and the short axis of the tumor were measured by using a Vernier caliper, and the tumor sizes were calculated according to an equation of ‘Tumor size=(long axis×(short axis)²)/2’. In FIGS. 8A and 8B, the negative control group administered PBS, the aPD1 group administered anti-PD1 antibodies as a positive control group, and the LMT19-32 group administered LMT19-32 as an experimental group. The graph of FIG. 8A shows tumor sizes according to a number of days after the cell injection, and FIG. 8B shows tumor sizes on day 21 after the cell injection. In FIG. 8A, the horizontal axis represents days after the tumor cell administration on which tumor sizes were measured. In FIG. 8B, the bars indicate all tumor sizes of each group on day 21 after the cell injection. As shown in FIGS. 8A and 8B, statistically significant tumor growth inhibition was observed in the group administered with LMT19-32 strain alone.

### 3. Analysis of Tumor-Infiltrating Immune Cells and Evaluation of Functionality of Immune Cells

CD8 T cells, which are tumor-infiltrating immune cells, are important markers of anti-cancer responses, and interferon gamma and granzyme B secreted by CD8 T cells are functional cytokines that indicate activity of immune cells. FIGS. 9A, 9B, and 9C show results of analyzing tumor-infiltrating T cells, and cytokine secretion thereby, after administration of the Enterococcus faecalis LMT19-32 strain to mice with tumor. The administration was performed according to Section 1. Tumors were excisedd 21 days after the administration. The excised tumors were separated into single cells by using RPMI1640 medium containing 50 μg/ml of liberase and 40 μg/ml of DNase I, and a cell strainer. In order to observe interferon gamma production pattern of T cells, the isolated cells were stimulated for 4 hours with 50 ng/ml of phorbol 12-myristate 13-acetate (PMA) and 500 ng/ml of ionomycin in RPMI1640 medium containing 10% FBS. PMA and ionomycin are substances that give signal stimulations for activating T cells, and bring the same results as an activation mechanism of T cells by actual antigens, creating an environment in which an immune response occurs. lonomycin increases protein kinase C (PKC) as a Ca²⁺ ionophore, and PMA plays a synergizing role by phosphorylating PKC to target CD4 T cells and CD8 T cells. After the stimulation, the cells were stained with the antibodies in Table 12 and analyzed by using CANTO II flow cytometry equipment for immune cell analysis and confirmation of interferon gamma production.

TABLE 12

| Description | Target | Color |
|---|---|---|
| Surface marker | CD45 | PE-Cy7 |
| | TCRβ | APC-Cy7 |
| | CD4 | PerCP-Cy5.5 |
| | CD8 | Pacific blue |
| Intracellular staining | IFNγ | FITC |
| | Granzyme B | APC |

FIGS. 9A, 9B, and 9C show total numbers of tumor-infiltrating T cells, numbers of CD8 T cells among tumor-infiltrating T cells, and numbers of IFNγ-expressing cells among CD8 T cells, respectively. As shown in FIG. 9, a number of tumor-infiltrating T cells and a number of CD8 T cells increased significantly in the group administered with LMT19-32 compared to the PBS-treated group. In addition, a number of CD8 T cells producing interferon gamma increased significantly in the LMT19-32-treated group compared to the PBS control group.

1. A method of preventing or treating tumor in a subject, comprising administrating a bacterial strain to a subject in need of prevention or treatment of tumor, wherein the bacterial strain
i) belongs to a species Enterococcus faecium;
ii) has a lactate production ability compared to microbial growth (lactate/OD₆₀₀) of 3 g/L or more, when cultured for 48 hours; and
iii) exhibits anti-tumor activity.

2. The method of claim 1, wherein the bacterial strain has lactate production ability (lactate/OD₆₀₀) compared to microbial growth that is greater than 2.5 g/L, when the bacterial strain is cultured for 24 hours.

3. The method of claim 1, wherein the bacterial strain exhibits a tumor inhibition rate of 10% or more.

4. The method of claim 1, wherein the bacterial strain exhibits a tumor inhibition rate of 20% or more.

5. The method of claim 1, wherein the bacterial strain has iv) ß-galactosidase activity.

6. The method of claim 1, wherein the bacterial strain has v) a D-sorbitol decomposition ability.

7. The method of claim 1, wherein the bacterial strain has vi) a D-tagatose decomposition ability.

8. The method of claim 1, wherein the bacterial strain has vii) a methyl-αD-mannopyranoside decomposition ability.

9. The method of claim 1, wherein the bacterial strain is at least one selected from the following groups:
LMT17-62 deposited under an accession number of KCTC 14284BP;
LMT17-74 deposited under an accession number of KCTC14285BP;
LMT15-24 deposited under an accession number of KCTC 14289BP;
LMT17-25 deposited under an accession number of KCTC 14288BP; and
LMT15-4 deposited under an accession number of KCTC 14290BP.

10. A composition for preventing or treating tumor comprising a bacterial strain, as an active ingredient, wherein the bacterial strain i) belongs to a species Enterococcus faecium;
ii) has a lactate production ability compared to microbial growth (lactate/OD₆₀₀) of 3 g/L or more, when cultured for 48 hours; and
iii) exhibits anti-tumor activity.

11. The composition of claim 10, wherein the bacterial strain is a lyophilized form or a dried form.

12. The composition of claim 11, wherein the composition is in the form of a capsule or a tablet.

13. The composition of claim 10, wherein the tumor is solid tumor.

14. The composition of claim 10, comprising the bacterial strain in an amount of 1×10⁶ CFU or more.

15. The composition of claim 10, for co-administrating with at least one other therapeutic agent.

16. The composition of claim 15, wherein the therapeutic agent is an immuno-anti-cancer agent.

17. The composition of claim 16, wherein immuno-anti-cancer agent is an immune checkpoint inhibitor.

18. The composition of claim 17, wherein the immune checkpoint inhibitor is a PD-1 antagonist, a PD-L1 antagonist, a CTLA-4 antagonist, or a combination thereof.

19. The composition of claim 18, wherein the immune checkpoint inhibitor is at least one selected from anti-PD-1 antibodies, anti-PD-L1 antibodies, anti-CTLA4 antibodies, or antigen-binding fragments thereof.

20. The composition of claim 14, for administrating with at least one other therapeutic agent concurrently or sequentially.

21. The method of claim 1, comprising: administrating a composition to a subject in need of prevention or treatment of tumor, wherein the composition comprising a bacterial strain, as an active ingredient, wherein the bacterial strain i) belongs to a species Enterococcus faecium;
ii) has a lactate production ability compared to microbial growth (lactate/OD₆₀₀) of 3 g/L or more, when cultured for 48 hours; and
iii) exhibits anti-tumor activity.
